(19) United States
(12) Patent Application Publication
(10) Pub. No.: US 2020/0261520 A1
(43) Pub. Date: Aug. 20, 2020

(54) Combination Therapies of Hepatitis B Virus (HBV)-infected individuals using Parapoxvirus Ovis (PPVO) and at Least one Further Antiviral Drug

(71) Applicant: AiCuris GmbH & Co. KG, Wuppertal (DE)

(72) Inventors: Daniela PAULSEN, Wuppertal (DE); Andreas URBAN, Sprockhovel (DE); Ibironke ADDY, Dusseldorf (DE); Tamara PFAFF, Dusseldorf (DE); Stephan MENNE, Alexandria, VA (US); Willem SLOOT, Darmstadt (DE)

(73) Assignee: AiCuris GmbH & Co. KG, Wuppertal (DE)

(21) Appl. No.: 16/645,020

(22) PCT Filed: Sep. 7, 2018
(86) PCT No.: PCT/EP2018/074202

§ 371 Date: Mar. 6, 2020

(30) Foreign Application Priority Data
Sep. 7, 2017 (EP) 17189890.1
Oct. 16, 2017 (EP) 17196684.9

(51) Int. Cl. A61K 35/76; C12N 7/00; A61K 39/39; A61K 45/06; A61P 31/20

(52) U.S. Cl. CPC A61K 35/76; C12N 7/00; A61K 39/39; A61K 2039/58; A61P 31/20; C12N 2710/24232; A61K 45/06

(57) ABSTRACT

The present invention relates to new combination therapies of HBV-infected individuals using a Parapoxvirus ovis (PPVO) and at least one further antiviral drug, e.g., nucleoside inhibitors, such as Entecavir. The methods and combination products according to the present invention are safe and suitable for the induction of a functional cure in chronically HBV-infected patients.

### TECHNICAL FIELD

The present invention relates to new combination therapies of HBV-infected individuals using a Parapoxvirus ovis (PPVO) and at least one further antiviral drug, e.g., nucleoside inhibitors, such as Entecavir. The methods and combination products according to the present invention are safe and suitable for the induction of a functional cure in chronically HBV-infected patients.

### BACKGROUND

Hepatitis B Virus (HBV) is an enveloped, partially double-stranded DNA (dsDNA) virus of the hepadnavirus family (Hepadnaviridae). Chronic HBV infection is a significant global health problem, affecting over 5% of the world population (over 350 million people worldwide and 1.25 million individuals in the US).

Despite the availability of a prophylactic HBV vaccine, the burden of chronic HBV infection continues to be a significant unmet worldwide medical problem, due to suboptimal treatment options and sustained rates of new infections in most parts of the developing world. Current treatments do not provide a cure and are limited to only two classes of agents (interferon alpha and nucleoside/nucleotide analogues/inhibitors of the viral polymerase); drug resistance, low efficacy, and tolerability issues limit their impact.

The low cure rates of HBV are attributed at least in part to the fact that complete suppression of virus production is difficult to achieve with a single antiviral agent, and to the presence and persistence of covalently closed circular DNA (cccDNA) in the nucleus of infected hepatocytes. However, persistent suppression of HBV DNA slows liver disease progression and helps to prevent hepatocellular carcinoma (HCC).

Current therapy goals for HBV-infected patients are directed to reducing serum HBV DNA to low or undetectable levels, and to ultimately reducing or preventing the development of cirrhosis and HCC.

Currently, the highest rate of functional cure in HBV occurs following therapy with the immunomodulator Interferon-alpha (IFN-alpha). But even treatment with 48-52 weeks of IFN-alpha results in responder rates of about 3-7% durable HBsAg loss at best.

Nevertheless, quantitative HBsAg measurements have been associated with response to treatment and have been shown to predict response: Low HBsAg titer (around or ≤100 IU/ml) at end of treatment with nucleoside or nucleotide inhibitors were associated with response or a lower risk of relapse after stopping therapy. In addition, quantitative HBsAg has also been shown to predict response to IFN-alpha treatment.

Consequently, decline or loss of HBsAg is used as predictor for functional cure and implemented in the international EASL HBV treatment guideline (EASL clinical practice guidelines April 2017).

There is a need to develop new strategies of treating HBV-infections and/or to achieve a functional cure in chronically HBV-infected individuals. This objective is addressed in the present invention using the best experimental animal model system to examine immune-mediated functional cure of HBV, i.e., woodchucks chronically infected with woodchuck hepatitis virus (WHV).

### SUMMARY OF THE INVENTION

Provided herein are combination products and methods for the treatment of HBV infection in a subject in need thereof. The subject matter of the invention is disclosed in the following embodiments.

Subject-matter of the invention is a composition comprising Parapoxvirus ovis selected from the group comprising:
optionally inactivated Parapoxvirus ovis (PPVO) virions and/or active fragments thereof, and/or
nucleic acid vectors or synthetic nucleic acid molecules expressing PPVO and/or at least one active fragment thereof, and/or
cells comprising PPVO virions or fragments thereof and/or nucleic acid vectors and/or synthetic nucleic acid molecules expressing PPVO and/or at least one active fragment thereof, for use in combination with at least one different antiviral drug for the treatment of an individual with a Hepatitis B Virus (HBV) infection.

As used herein, it is possible to first use either the at least one antiviral drug (e.g., a nucleoside/nucleotide analogue, such as Entecavir, Tenofovir, etc.) or to use PPVO first, and subsequently (secondly) treat the individual in need thereof with PPVO in combination with the at least one additional antiviral drug, and further subsequently (thirdly) continue the treatment with PPVO as disclosed herein either for a short period (days to weeks) or for months or even years ad infinitum as maintenance therapy. Alternatively, the treatment may be continued with at least one antiviral drug. Additionally, the combination treatment according to the present invention may be repeated as required, e.g. at least once, twice, three times, etc. In addition, combination therapy can be started at the same time and/or stopped at the same time.

Subject-matter of the invention is the composition comprising Parapoxvirus ovis selected from the group comprising:
optionally inactivated Parapoxvirus ovis (PPVO) virions and/or active fragments thereof, and/or
nucleic acid vectors or synthetic nucleic acid molecules expressing PPVO and/or at least one active fragment thereof, and/or
cells comprising PPVO virions or fragments thereof and/or nucleic acid vectors and/or synthetic nucleic acid molecules expressing PPVO and/or at least one active fragment thereof,
   for use in combination with at least one different antiviral drug for the treatment of an individual with a Hepatitis B Virus (HBV) infection in accordance with the preceding embodiment, wherein the different antiviral drug is an anti-HBV antiviral drug.

Subject-matter of the invention is the composition comprising Parapoxvirus ovis (PPVO) for use in combination with at least one different antiviral drug for the treatment of an individual with a Hepatitis B Virus (HBV) infection in accordance with the above embodiments of the invention, wherein the PPVO is a recombinant virus nucleic acid or at least one active fragment thereof, and/or wherein the PPVO is a recombinantly produced virion and/or at least one active fragment thereof.

Subject-matter of the invention is the composition comprising Parapoxvirus ovis (PPVO) for use in combination with at least one different antiviral drug for the treatment of an individual with a Hepatitis B Virus (HBV) infection according to any one of the preceding embodiments, wherein the PPVO is selected from the group of PPVO strains comprising NZ2, NZ7, NZ10, D1701, OV/20, OV/7, OV/C2, OV/mi-90, OV-Torino, SA00, Bo29, orf11, Greek orf strain 155, and/or Greek orf strain 176, or a taxonomically related Parapoxvirus ovis orf strain.

Subject-matter of the invention is the composition comprising PPVO for use in combination with at least one different antiviral drug for the treatment of an individual with a HBV infection according to any one of the preceding embodiments, wherein the antiviral drug is selected from the group of drugs comprising nucleotide/nucleoside analogues as active ingredients, Capsid assembly inhibitors or modulators, capsid/core inhibitors or modulators, encapsidation inhibitors or modulators, RNAi, Therapeutic vaccination, Toll-like-receptor (TLR) agonists and antagonists, epigenetic modifiers, entry inhibitors or modulators, cyclophilin inhibitors or modulators, Inhibitors of HBsAg secretion, HBsAg inhibitors, HBV entry inhibitors or modulators, cccDNA inhibitors, immunomodulators (Interferons and other cytokines), and/or check-point inhibitors (e.g. PD-1).

Subject-matter of the invention is the composition comprising PPVO for use in combination with at least one different antiviral drug for the treatment of an individual with a HBV infection according to any one of the preceding embodiments, wherein the group of drugs comprising nucleotide/nucleoside analogues as active ingredients comprises Tenofovir, Tenofovir disoproxil fumarate (TDF), Tenofovir-Alafenamid (TAF), Entecavir, Lamivudine, Telbivudine, Adefovir, Emtricitabine and/or Clevudine.

Subject-matter of the invention is the composition comprising PPVO for use in combination with at least one different antiviral drug for the treatment of an individual with a HBV infection according to any one of the preceding embodiments, wherein PPVO and the at least one different antiviral drug are formulated for separate/subsequent administration, or wherein the PPVO and the at least one different drug as defined in any of the preceding embodiments are formulated for concomitant/simultaneous administration.

Subject-matter of the invention is the composition comprising PPVO for use in combination with at least one different antiviral drug for the treatment of an individual with a HBV infection according to any one of the preceding embodiments, wherein PPVO and the at least one different antiviral drug are formulated for separate/subsequent administration, or wherein the PPVO and the at least one different drug as defined in any of the preceding embodiments s are formulated for concomitant/simultaneous administration.

Subject-matter of the invention is the composition comprising PPVO for use in combination with at least one different antiviral drug for the treatment of an individual with a HBV infection according to any one of the preceding embodiments, wherein the PPVO and the at least one different antiviral drugs are provided as single drug units or combination products selected from the group comprising: tablets, capsules, lozenges, particularly acid-resistant capsules, drops, patches, depot administration forms, solutions, solutions for injection, solution for infusion, dilutions, creams, ointments, salves, powders, powder for reconstitution, powder for reconstitution and infusion, and/or sprays.

Subject-matter of the invention is the composition comprising PPVO for use in combination with at least one different antiviral drug for the treatment of an individual with a HBV infection according to any one of the preceding embodiments, wherein said individual is selected from the group of patients with acute HBV infection, chronic HBV infection, patients with detectable HBsAg, patients with detectable HBV RNA, patients with detectable HBV DNA, patients with detectable cccDNA, patients with liver inflammation, patients with liver steatosis, patients with liver fibrosis, patients with liver cirrhosis, patients with liver cancer, patients with hepatocellular carcinoma, acutely or asymptomatically or chronically infected patients, patients subjected to antiviral treatment, patients that do not respond to antiviral treatment with antiviral drugs according to any one of the preceding embodiments, or patients that have acquired resistance to at least one antiviral drug, and/or patients that are co-infected with at least one additional pathogenic virus selected from the group comprising deltavirus, retroviridae, herpesviridae, poxviridae, parvoviridae, adenoviridae, picornaviridae, hepadnaviridae, flaviviridae, orthomyxoviridae, paramyxoviridae, papovaviridae, polyomaviridae, rhabdoviridae, coronaviridae, bunyaviridae, arenaviridae, reoviridae, and togaviridae.

Subject-matter of the invention is the composition comprising PPVO for use in combination with at least one different antiviral drug for the treatment of an individual with a HBV infection according to any one of the preceding embodiments, wherein the dose of PPVO is in the range of 1×10⁶ 1×10¹⁰ viral particles and the dose of the at least one different antiviral drug is selected according to the manufacturer's instructions.

Subject-matter of the invention is the composition comprising PPVO for use in combination with at least one different antiviral drug for the treatment of an individual with a HBV infection as defined in the preceding embodiments, wherein PPVO and the at least one different antiviral drug are administered for ≤72 weeks, preferably ≤60 weeks, more preferably ≤48 weeks, ≤36 weeks, ≤24 weeks, ≤12 weeks, ≤6 weeks, ≤4 weeks, ≤2 weeks, or ≤1 week.

Subject-matter of the invention is the composition according to any of the preceding embodiments for use in combination with at least one different antiviral drug for the treatment of an individual with HBV infection according to any one of the preceding embodiments, wherein the PPVO is inactivated.

Subject-matter of the invention is the composition comprising PPVO for use in combination with at least one different antiviral drug for the treatment of an individual with a HBV infection according to any one of the preceding embodiments, wherein the at least one different antiviral drug is Entecavir.

Subject-matter of the invention is the composition comprising PPVO for use in combination with at least one different antiviral drug for the treatment of an individual with a HBV infection according to any one of the preceding embodiments, wherein the patient treated with said composition and with at least one different antiviral drug is a patient that is HBsAg and/or HBeAg positive, and wherein the HBsAg and/or HBeAg load is reduced or HBsAg and/or HBeAg loss occurs over the course of the treatment as defined in any of the foregoing embodiments.

Subject-matter of the invention is the composition comprising PPVO for use in combination with at least one different antiviral drug for the treatment of an individual with a HBV infection according to any one of the preceding embodiments, wherein the composition is formulated for intravenous, intramuscular, oral, parenteral, intradermal, and/or subcutaneous administration.

Subject-matter of the invention is a method of treatment of a HBV-infected patient in need thereof with an effective amount of PPVO and an effective amount of at least one different antiviral drug, wherein the PPVO is selected from the group comprising:
optionally inactivated Parapoxvirus ovis (PPVO) virions and/or active fragments thereof, and/or
nucleic acid vectors or synthetic nucleic acid molecules expressing PPVO and/or at least one active fragment thereof, and/or
cells comprising PPVO virions or fragments thereof and/or nucleic acid vectors and/or synthetic nucleic acid molecules expressing PPVO and/or at least one active fragment thereof. Subject-matter of the invention is the method of treatment according to the preceding embodiment, wherein the PPVO is a recombinant virus nucleic acid or at least one active fragment thereof, and/or wherein the PPVO is a recombinantly produced virion and/or active fragments thereof.

Subject-matter of the invention is the method according to the preceding embodiments, wherein the different antiviral drug is selected from wherein the antiviral drug is selected from the group of drugs comprising nucleotide analogues as active ingredients, Capsid assembly inhibitors or modulators, capsid/core inhibitors or modulators, encapsidation inhibitors or modulators, RNAi, Therapeutic vaccination, Toll-like-receptor (TLR) agonists and antagonists, epigenetic modifiers, entry inhibitors or modulators, cyclophilin inhibitors or modulators, Inhibitors of HBsAg secretion, HBsAg inhibitors, HBV entry inhibitors or modulators, cccDNA inhibitors, immunomodulators (e.g., Interferons and other cytokines), and/or check-point inhibitors (e.g. PD-1),

Subject-matter of the invention is the method according to any one of the preceding embodiments, wherein the group of drugs comprising nucleotide/nucleotides analogues as active ingredients comprises Tenofovir, Tenofovir disoproxil fumarate (TDF), Tenofovir-Alafenamid (TAF), Entecavir, Lamivudine, Telbivudine, Adefovir, Emtricitabine, and/or Clevudine.

Subject-matter of the invention is the method according to any one of the preceding embodiments, wherein the antiviral drug is Entecavir.

Subject-matter of the invention is the method according any one of the preceding embodiments, wherein PPVO and the at least one different antiviral drug are separately/separately administered.

Subject-matter of the invention is the method according any one of the preceding embodiments, wherein PPVO and the at least one different antiviral drug are concomitantly/simultaneously administered.

Subject-matter of the invention is the method according any one of the preceding embodiments, wherein PPVO and the at least one different antiviral drug is provided in separate single unit form or as a combination products selected from the group comprising: tablets, capsules, lozenges, particularly acid-resistant capsules, drops, patches, depot administration forms, solutions, solutions for injection, solution for infusion, dilutions, creams, ointments, salves, powders, powder for reconstitution, powder for reconstitution and infusion, and/or sprays.

Subject-matter of the invention is the method according any one of the preceding embodiments, wherein PPVO and/or the at least one different antiviral drug are formulated for intravenous, intramuscular, oral, parenteral, topical, intradermal, and/or subcutaneous administration.

Subject-matter of the invention is the method according to any one of the preceding embodiments, wherein said individual is selected from the group of patients with acute HBV infection, chronic HBV infection, patients with detectable HBsAg, patients with detectable HBV RNA, patients with detectable HBV DNA, patients with detectable cccDNA, patients with liver inflammation, patients with liver steatosis, patients with liver fibrosis, patients with liver cirrhosis, patients with liver cancer, patients with hepatocellular carcinoma, asymptomatic or acutely or chronically infected patients, patients subjected to antiviral treatment, patients that do not respond to antiviral treatment with antiviral drugs according to any one of the preceding embodiments, or patients that have acquired resistance to at least one antiviral drug, and/or patients that are co-infected with at least one additional pathogenic virus selected from the group comprising deltavirus, retroviridae, herpesviridae, poxviridae, parvoviridae, adenoviridae, picornaviridae, hepadnaviridae, flaviviridae, orthomyxoviridae, paramyxoviridae, papovaviridae, polyomaviridae, rhabdoviridae, coronaviridae, bunyaviridae, arenaviridae, reoviridae, and togaviridae.

Subject-matter of the invention is the method according to any one of the preceding embodiments, wherein the dose of PPVO is in the range of 1×10⁶ 1×10¹⁰ viral particles, and/or wherein the dose of the at least one different antiviral drug is selected according to the manufacturer's instructions.

Subject-matter of the invention is the method according to any one of the preceding embodiments, wherein PPVO and the at least one different antiviral drug are administered for ≤72 weeks, preferably <60 weeks, more preferably <48 weeks, <36 weeks, <24 weeks, <12 weeks, <6 weeks, <4 weeks, <2 weeks, or <1 week.

Subject-matter of the invention is a method for the reduction of HBV viral load in a HBV-infected patient in need thereof comprising administering an effective amount of PPVO and an effective amount of at least one different antiviral drug as defined in any of the foregoing embodiments.

Subject-matter of the invention is a method for the reduction of HBsAg load in a HBV-infected patient in need thereof comprising administering an effective amount of PPVO and an effective amount of at least one different antiviral drug as defined in any of the foregoing embodiments.

Subject-matter of the invention is a method for the reduction of liver damage, liver cirrhosis, and/or liver fibrosis, in a HBV-infected patient in need thereof comprising administering an effective amount of PPVO and an effective amount of at least one different antiviral drug as defined in any of the foregoing embodiments.

Subject-matter of the invention is a method for inducing liver tissue regeneration in a HBV-infected patient in need thereof comprising administering an effective amount of PPVO and an effective amount of at least one different antiviral drug as defined in any of the foregoing embodiments.

Subject-matter of the invention is a method for reducing side-effects associated with the treatment of a HBV-infected patient, wherein said side-effects are caused by the treatment with interferons and/or nucleotide/nucleoside analogues comprising administering an effective amount of PPVO and an effective amount of at least one different antiviral drug as defined in any of the foregoing embodiments, in particular the reduction of side-effects selected from the group comprising fever, tissue inflammation, psychological disturbances, and/or hematological disturbances.

Subject-matter of the invention is a method for the reduction of HBeAg load in a HBV-infected patient comprising administering an effective amount of PPVO and an effective amount of at least one different antiviral drug as defined in any of the foregoing embodiments.

Subject-matter of the invention is a method for the restoration and/or reactivation of the immune response in a HBV-infected patient in need thereof comprising administering an effective amount of PPVO and an effective amount of at least one different antiviral drug as defined in any of the foregoing embodiments.

Subject-matter of the invention is a method of reducing the amount of HBV DNA, eliminating HBV DNA and/or silencing HBV DNA, in particular cccDNA in a HBV-infected patient comprising administering an effective amount of PPVO and an effective amount of at least one different antiviral drug as defined in any of the foregoing embodiments.

Subject-matter of the invention is a method of preventing the de novo formation of cccDNA in a HBV-infected patient comprising administering an effective amount of PPVO and an effective amount of at least one different antiviral drug as defined in any of the foregoing embodiments.

Subject-matter of the invention is a method of inhibiting or reducing the expression of HBV proteins in a HBV-infected patient comprising administering an effective amount of PPVO and an effective amount of at least one different antiviral drug as defined in any of the foregoing embodiments.

Subject-matter of the invention is a method of suppressing replication of HBV in a HBV-infected patient comprising administering an effective amount of PPVO and an effective amount of at least one different antiviral drug as defined in any of the foregoing embodiments.

Subject-matter of the invention is a method of eradication of HBV in a HBV-infected patient comprising administering an effective amount of PPVO and an effective amount of at least one different antiviral drug as defined in any of the foregoing embodiments.

Subject-matter of the invention is a method of breaking immunological tolerance towards HBV infections in a HBV-infected patient comprising administering an effective amount of PPVO and an effective amount of at least one different antiviral drug as defined in any of the foregoing embodiments.

Subject-matter of the invention is a method of breaking tolerance towards HBsAg and/or HBeAg in a HBV-infected patient comprising administering an effective amount of PPVO and an effective amount of at least one different antiviral drug as defined in any of the foregoing embodiments.

Subject-matter of the invention is a method of inducing HBsAg-specific antibodies in a HBV-infected patient comprising administering an effective amount of PPVO and an effective amount of at least one different antiviral drug as defined in any of the foregoing embodiments.

Subject-matter of the invention is a method of inducing HBeAg-specific antibodies in a HBV-infected patient comprising administering an effective amount of PPVO and an effective amount of at least one different antiviral drug as defined in any of the foregoing embodiments.

Subject-matter of the invention is a method of slowing down or inhibiting the progression of steatosis in a HBV-infected patient comprising administering an effective amount of PPVO and an effective amount of at least one different antiviral drug as defined in any of the foregoing embodiments.

Subject-matter of the invention is a method according to any one of the preceding embodiments, wherein PPVO and the at least one different antiviral drug are administered for <72 weeks, preferably <60 weeks, more preferably <48 weeks, <24 weeks, <12 weeks, <6 weeks, <4 weeks, <2 weeks, or <1 week.

Subject-matter of the invention is a medicinal kit product comprising a first container comprising a pharmaceutical compositions comprising PPVO, preferably inactivated PPVO, and a second container comprising a pharmaceutical compositions comprising at least one different antiviral drug as defined in any one of the preceding embodiments or a pharmaceutical composition comprising PPVO, preferably inactivated PPVO, and at least one different antiviral drug as defined in any one of the preceding embodiments in form of a combined formulation, and optionally instructions for use, pharmaceutically acceptable media for reconstitution, syringes, and/or microneedles, etc.

Subject-matter of the invention is a medicinal kit product according to the preceding embodiment, wherein the compositions comprising PPVO, preferably inactivated PPVO, and the pharmaceutical composition comprising at least one different antiviral drug are formulated as tablets, capsules, lozenges, particularly acid-resistant capsules, drops, patches, depot administration forms, solutions, solutions for injection, solution for infusion, dilutions, creams, ointments, salves, powders, powder for reconstitution, powder for reconstitution and infusion, and/or sprays, etc.

EXPERIMENTS

In the woodchuck animal model of chronic hepatitis B, intramuscular treatment with the immunomodulator AIC649 for 8 weeks has been shown to induce a unique bi-phasic response pattern, with reduction of woodchuck hepatitis virus (WHV) DNA and surface antigen (WHsAg) (Paulsen et al. (2015)).

In the present example, the antiviral activity of AIC649, alone or in combination with Entecavir (ETV), as well as different dosing routes and longer treatment periods were explored in the woodchuck. The objective was to further explore the safety and potential of AIC649 to induce a functional cure in chronic hepatitis B virus infection.

In woodchucks, over a 36-week period, AIC649 was administered intravenously and then intramuscularly, alone or in combination with an initial 12 weeks of the direct acting antiviral, ETV, given orally. The efficacy of AIC649 monotherapy, ETV monotherapy, or combination AIC649+ETV therapy was compared to a placebo control group.

Treatment-induced changes in viremia, antigenemia, immunological parameters, as well as the induction of WHsAg antibody seroconversion were evaluated for determination of antiviral effects. Daily observations, changes in body weight and body temperatures, changes in hematology and clinical chemistry, as well as necropsy and histopathology were assessed for determination of safety.

The bi-phasic response pattern induced by AIC649 monotherapy previously observed was confirmed. Treatment with AIC649 alone already led to a clear reduction of WHV DNA as well as WHsAg from pretreatment levels. A significant and even stronger and sustained antiviral effect was observed in the AIC649+ETV combination group: WHV DNA and WHsAg stayed markedly suppressed or even undetectable for several months. Cell mediated immune responses, as well as anti-WHsAg antibody response, were observed in the two groups receiving AIC649 but not in the ETV monotherapy group. The changes in the liver disease markers were comparable between the groups, but the progression of steatosis during the study appeared slower in the AIC649 alone and the AIC649+ETV combination group. The observed sustained loss of WHsAg and the induction of anti-WHsAg antibodies accompanied by cell mediated immune responses support the hypothesis that AIC649 induces a physiologically “concerted”, reconstituted immune response to WHV. AIC649 as a combination partner to ETV dramatically increases the efficacy of treatment. AIC649's potential for inducing a functional cure in HBV-infected patients is surprisingly supported by this preclinical study.

Experiment 1

In the present study, the antiviral activity of AIC649, alone or in combination with Entecavir (ETV), as well as different dosing routes and longer treatment periods were explored in chronically WHV-infected woodchucks. The objective was to further explore the safety and potential of AIC649 to induce a functional cure in chronic hepatitis B virus infection.

Methods

In woodchucks, over a 36-week period, AIC649 was administered i.v. and then i.m., alone or in combination with an initial 12 weeks of the direct acting antiviral, ETV, given orally. See FIG. 1: Study design

The efficacy of AIC649 monotherapy, ETV monotherapy, or combination AIC649+ETV therapy was compared to a placebo control group (N=5 animals/group). Treatment induced changes in viremia, antigenemia, immunological parameters, as well as the induction of WHsAg antibody seroconversion were evaluated for determination of antiviral effects. Daily observations, changes in body weight and body temperatures, changes in hematology and clinical chemistry, as well as necropsy and histopathology were assessed for determination of safety.

Animals

All 20 woodchucks (10 males, 10 females) used in this study were born in captivity were inoculated at 3 days of age with the cWHV7P2a inoculum, containing woodchuck hepatitis virus (WHV) strain WHV7-11 (Fletcher et al. (2015): PLOSPath, 11, e1005103). Pups inoculated with cWHV7P2a were reared and maintained until they were approximately 15 to 17 months of age. Woodchucks were housed in pairs or individually and given ad libitum access to food and water throughout the pre-study and study periods. All woodchucks received orally, daily 50 mg/kg of fenbendazole (panacur) for 10 days for treatment of possible infection with Giardia. Woodchucks were screened for biochemical and hematologic abnormalities. Woodchucks were also tested for WHsAg, antibodies against WHsAg (anti-WHs) and WHV DNA. All woodchucks were confirmed as established chronic carriers of WHV based on accepted criteria developed over the past 30 years of experience with neonatal experimental infections with WHV (i.e., WHV DNA and WHsAg positive, anti-WHs negative at 9 to 12 months of age and thereafter). Absence of liver tumors in woodchucks with low serum gamma-glutamyl transferase (GGT) activity was confirmed by ultrasonography in 16 of the 20 woodchucks received. Three other woodchucks had slightly to moderately elevated serum GGT activity, but presented without liver tumors during the initial ultrasonography. All woodchucks were implanted with microchips (dorsal base of the tail) that had been programmed with the animal identification number (DASHost Software, Biomedical Data Systems Inc.). Animals were manually randomized to 1 of 4 treatment groups, stratified by gender, body weight, pre-treatment serum markers (WHsAg, WHV DNA levels, anti-WHsAg antibody titers), complete blood counts (CBCs), serum levels of GGT and sorbitol dehydrogenase (SDH).

### AIC649 (and Vehicle AIC)

AIC649: Lyophilisate for reconstitution, in glass vials with 1.1×10⁹ viral particles (parapox virus ovis (PPVO))/vial, reconstituted in 1.1 mL pyrogen-free water
Vehicle AIC: Lyophilisate for reconstitution, reconstituted in 1.1 mL pyrogen-free water
Method of administration: Intravenous or intramuscular
Frequency of administration: Weeks 1 to 24: twice weekly
   Weeks 25 to 36: once weekly
Dose levels: AIC649: 1×10⁹ viral particles (PPVO)
   Vehicle: 0 viral particles (PPVO)
Dose volume: 1 mL
Duration of treatment Total: 36 weeks (12 weeks i.v., followed by 24 weeks i.m.)

### ETV (and Vehicle ETV)

Entecavir (ETV): Powder (Selleckchem (Munich, Germany), suspended in sterile isotonic saline
Vehicle ETV: sterile isotonic saline
Method of administration: Oral
Frequency of administration: Week 1 to 12: daily
Dose levels: ETV: 0.2 mg/kg
   Vehicle: 0 mg/kg
Dose volume: 0.2 mL ETV (or vehicle)+3 to 5 mL woodchuck liquid diet
Duration of treatment Total: 12 weeks

### Animal Procedures

Procedures were performed according to the schedule described in table 1:

TABLE 1
Schedule of procedures and assays

| | | Animal procedures | | | Assays | | | | |
|---|---|---|---|---|---|---|---|---|---|
| | | Body weight & | | Liver | WHV DNA & | Hematology & | | T cell | Cell markers |
| Study phase | Week | temperature | Bleed | biopsy^{a} | serology^{b} | biochemistry | aPTT & PT | response^{c} | & cytokines^{d} |
| Pretreatment | −2 to −1 | x | x | x | x | x | | | |
| Treatment | T = 0 | x | x | | x | | x | x | |
| | 1 | x | x | | x | | | | |
| | 2 | x | x | | x | | | | |
| | 3 | x | x | | x | | | | |
| | 4 | x | x | | x | | | | |
| | 5 | x | x | | x | | | | |
| | 6 | x | x | x | x | x | | x | x |
| | 7 | x | x | | x | | | | |
| | 8 | x | x | | x | | | | |
| | 9 | x | x | | x | | | | |
| | 10 | x | x | | x | | | | |
| | 11 | x | x | | x | | | | |
| | 12 | x | x | x | x | x | x | x | x |
| Treatment | 13 | x | x | | x | | | | |
| (2x/week) | 14 | x | x | | x | | | | |
| | 15 | x | x | | x | | | | |
| | 16 | x | x | x | x | x | | | |
| | 17 | x | x | | x | | | | |
| | 18 | x | x | | x | | | | |
| | 19 | x | x | | x | | | | |
| | 20 | x | x | | x | | | | |
| | 21 | x | x | | x | x | x | x | x |
| | 22 | x | x | | x | | | | |
| | 23 | x | x | | x | | | | |
| | 24 | x | x | x | x | x | x | x | x |
| Treatment | 25 | x | | | | | | | |
| (1x/week) | 26 | x | x | | x | | | | |
| | 27 | x | | | | | | | |
| | 28 | x | x | | x | | | | |
| | 29 | x | | | | | | | |
| | 30 | x | x | | x | x | x | x | x |
| | 31 | x | | | | | | | |
| | 32 | x | x | | x | | | | |
| | 33 | x | | | | | | | |
| | 34 | x | x | | x | | | | |
| | 35 | x | | | | | | | |
| | 36 | x | x | x | x | x | x | x | x |

aPTT = activated partial thromboplastin time;
DNA = deoxyribonucleic acid;
PT = prothrombin time;
WHV = woodchuck hepatitis virus
^{a}Liver biopsy analysis included: WHV DNA RI, WHV cccDNA, WHV RNA (Southern and Northern blots), histology, WHcAg, WHsAg, anti-WHsAg-Ab
^{b}DNA and serology included: WHV DNA (Slot blot & PCR), WHsAg, anti-WHsAg-Ab
^{c}T cell responses determined following stimulation of PBMCs with peptides covering entire WHsAg, WHcAg, WHVxAg
^{d}Samples stored at −80° C. for analysis of cell differentiation markers and cytokines.

Mortality was assessed daily for all animals.
Body weight/temperature and clinical signs were assessed weekly.
Blood samples were taken for serology during the pre-treatment period, weekly from randomization (T0) to Week 24 and every second week from Week 25 to Week 36.
Blood samples were taken for hematology and clinical chemistry during pre-treatment, at T0, and at Weeks 4, 8, 12, 16, 21, 24, 30 and 36.
Blood samples were taken for aPTT and PT assessment at T0, and Weeks 12, 21, 24, 30, and 36.
Blood samples were taken for assessment of T-cell responses at T0, and Weeks 6, 12, 21, 24, 30 and 36.
Blood samples were taken for assessment of cell differentiation markers and cytokine measurement during pre-treatment and at Week 6, 12, 21, 24, 30, and 36. Blood collection during the treatment period was performed approximately 16 hours after the administration of “Vehicle ETV”, “Vehicle AIC”, ETV, and/or AIC649
Liver biopsies were taken during the 2-week pre-treatment period, and at Weeks 6, 12, 16, 24, and 36.

Timing of Blood Sampling During the Treatment Period

Blood samples for analysis of cytokine and cellular markers were taken approximately 16 h post-treatment.

Blood samples for analysis of serology, virology, hematology (including aPTT and PT) or clinical chemistry, and T-cell responses were taken approximately 0.5 to 1.0 h after oral dosing (ETV/Vehicle ETV) and prior to i.v. dosing (AIC649/Vehicle AIC) to allow blood collection and iv dosing to be performed during the same anesthesia event. For intravenous injections of AIC649 or Vehicle AIC, animals were anesthetized using ketamine/xylazine i.m. injection, 10:1 mixture, 25 to 50 mg/kg ketamine/1.0 to 5.0 mg/kg xylazine, and/or isoflurane inhalation (ie, 1 to 3% via nose cone). Intramuscular injections of AIC649 or AIC Vehicle were performed without anesthesia.

Clinical Observations and Laboratory Investigations

Clinical Observations Signs

Mortality

Mortality was assessed daily for all animals.

Body temperature and weight

Body temperature and weight assessments were performed together, weekly. The animal identification number and body temperature were read from a microchip implanted in the dorsal base of the tail using a hand-held chip reader (Biomedical Data Systems Inc.). Data were reported as individual values and summarized by group as mean±SD.

Hematology

Uncoagulated whole blood was collected in EDTA tubes. Samples were shipped on cold packs for same-day delivery to a laboratory for analysis. Samples were analyzed the following day using an automated cell counter system (coulter) with program settings for woodchucks (Bellezza, C. A., et al., 2002, Elsevier. p. 309-328).

Complete blood counts included the following parameters:

| | |
|---|---|
| White blood cells (WBC) | Hematocrit (Hct) |
| Segmented Neutrophils | Mean cell volume (MCV) |
| Banded Neutrophils | Mean cell hemoglobin (MCH) |
| Lymphocytes | Mean cell hemoglobin concentration |
| | (MCHC) |
| Monocytes | Red cell distribution width (RDW) |
| Eosinophils | Platelet count (PLT) |
| Basophils | Mean platelet volume (MPV) |
| Red blood cells (RBC) | Reticulocytes (RETI) |
| Hemoglobin (Hb) | |

Clinical Chemistry

Serum was collected and frozen for shipping on dry ice within the sample week. Samples were thawed once for analysis. aPTT and PT were determined on citrate plasma. Samples were analyzed using an autoanalyzer (Hitachi) using established clinical chemistry assays for woodchucks (Peek, S. F., et al.: Hepatology, 2001. 33(1): p. 254-66).

The following clinical chemistry parameters were assessed:

| | |
|---|---|
| Alanine aminotransferase (ALT) | Albumin |
| Aspartate aminotransferase (AST) | Globulin |
| Sorbitol dehydrogenase (SDH) | Albumin/globulin ration (A/G ratio) |
| Alkaline phosphatase (ALP) | Activated partial thromboplastin time |
| | (aPTT) |
| Gamma-glutamyltransferase (GGT) | Prothrombin time (PT) |
| Glutamate dehydrogenase (GLDH) | Glucose |
| Sodium | Total bilirubin (T. bili) |
| Potassium | Direct bilirubin (D. bili) |
| Chloride | Indirect bilirubin (In. bili) |
| Bicarbonate | Amylase |
| Anion gap (AG) | Cholesterol |
| Na/K ratio | Creatine kinase (CK) |
| Urea | Iron (Fe) |
| Creatinine | Total iron binding capacity (TIBC) |
| Calcium | % saturation (of ferritin) |
| Phosphate | Lipemia |
| Magnesium | Hemolysis |
| Total protein (TP) | Icterus |

Histopathology

Tissues from animals terminated were fixed in 10% phosphate buffered formalin and sent for histopathologic al assessment.

A selection of the organ and tissue samples collected and fixed at necropsy were processed, wax paraffin embedded, cut at a nominal thickness of approximately 2 to 4 micrometers, and stained with hematoxylin and eosin. The slides were processed and stained and were examined by light microscope.

Organs Assessed

The following organs were collected for histological assessment:

adrenal glands, aorta, bone (femur) and articulation, bone (sternum) with bone marrow, bone marrow smears (fixed in methanol and stained by May Grunwald-Giemsa method), brain, bronchi (mainstem), caecum, colon, duodenum, epididymides, eyes, gallbladder, heart, ileum, injection site(s) (a sample was taken from the area injected), jejunum, kidneys and ureters, larynx, liver, lungs, lymph node (mandibular), lymph node (mesenteric), mammary gland, oesophagus, optic nerves, ovaries and oviducts, pancreas, parathyroid glands, Peyer's patches, pituitary gland, prostate, rectum, salivary glands (mandibular, parotid, sublingual), sciatic nerves, seminal vesicles, skeletal muscle, skin, spinal cord (cervical, thoracic, lumbar), spleen, stomach, testes, thymus, thyroid glands, tongue, trachea, ureters, urinary bladder, uterus (horns+cervix), vagina, all gross lesions.

Efficacy/Virological Assessments

WHV Antigen/Antibodies in Serum

Serum WHV DNA

WHV DNA levels in serum were quantified as genomic equivalents (ge's) using slot-blot hybridization with a standardized 32P-labelled WHV DNA fragment probe on a nitrocellulose membrane. The Lower Limit Of Quantification (LLOQ) of the assay was approximately 10⁷ ge/mL serum. Samples below the slot-blot LLOQ were also assessed using a quantitative real-time PCR assay (Menne, S., et al., Antimicrob Agents Chemother, 2008. 52(10): p. 3617-32) with an LLOQ approximately 5.0 to 7.0×10² WHV ge/mL serum.

Serum WHsAg

WHsAg levels in serum were quantified using an established ELISA with an LLOQ of approximately 5 ngWHsAg/mL serum (Cote, P. J., et al., Viral Immunol, 1993. 6(2): p. 161-9).

Serum Anti-WHs Antibodies

Anti-WHs antibody levels in serum were quantified using an established ELISA technique (Cote, P. J., et al., Viral Immunol, 1993. 6(2): p. 161-9). The LLOQ of the assay using a 1:100 sample dilution was approximately 100 StdU/mL serum. Samples were graded as follows:
100 to 200 StdU/mL were considered trace levels
200 to 300 StdU/mL were considered very low levels
300 to 500 StdU/mL were considered low levels
500 to 2000 StdU/mL were considered moderate levels
>2000 StdU/mL were considered high levels (e.g. as might be expected after 3-fold immunization of nave woodchucks with WHsAg-alum vaccine)

Care should be taken interpreting the results from this assay since in untreated chronically infected animals, there is a considerable excess of circulating WHsAg, making detection of unbound anti-WHs Ab unlikely. Therefore, negative results for anti-WHsAg detection demonstrate a lack of unbound WHs-specific antibody, not a lack of total WHs-specific antibody.

T-Cell Proliferative Responses

PBMCs were isolated from whole blood and cultured for 5 days in the presence of DMSO (negative control), lipopolysaccharide (LPS, positive control), recombinant human IL-2 (positive control), or pools of peptides covering the entire WHcAg, WHsAg, or WHxAg, respectively. Using the CellTiter-Glo® assay, the number of viable cells in each test well was assessed using a luminescent signal proportional to the amount of ATP present, and thus to the number of viable cells. Cultures were assessed after 5 days and a stimulation index (SI) was derived relative to the negative controls as a measure of T-cell responses to specific antigens/stimuli:

SI=Test luminescence/negative control luminescence

Cytokines and Cell Markers

Immune responses associated with treatment were determined by changes in RNA transcript levels of IFN-α, IFN-γ, TNF-α, interleukin 6 (IL-6), CD3, CD4, CD8, CD14, CD56, and CD79B in blood and liver using PCR. Briefly, whole blood was collected into PAXgene blood tubes (Qiagen, Redwood City, Calif.) at time points indicated above. Samples were stored at −70° C. until use. Total RNA was further isolated from liver biopsy samples collected as indicated above using the RNeasy Mini Kit (Qiagen) with on-column DNase I digestion using RNase-Free DNase (Qiagen).

Following reverse transcription of mRNA with the High Capacity cDNA Reverse Transcription Kit (Applied Biosystems) using oligo(dT), complementary (c) DNA samples were amplified on a 7500 Real Time PCR System instrument (Applied Biosystems) using TaqMan Gene Expression Master Mix (Applied Biosystems) and woodchuck-specific primers and probes (Table 2). Woodchuck 18S rRNA expression was used to normalize target gene expression. Transcription levels of target genes were calculated as a fold-change relative to pretreatment level at week −1 (liver) or at T0 (blood) using the formula 2^{ΔCt}.

TABLE 2
Oligonucleotides used for analyses of gene expression in blood
and liver

| Gene | Primers and Probe | Sequence |
|---|---|---|
| IFN-α | F (SEQ ID NO: 1) | 5'-CTCAAGCTGTTGCTGTCCTC-3' |
| | R (SEQ ID NO: 2) | 5'-CTTCTGGGTGCTGAAGAGGT-3' |
| | P (SEQ ID NO: 3) | 5'-CCAGATGACCCAGCAGATCCTCA-3' |
| IFN-γ | F (SEQ ID NO: 4) | 5'-ATCCAAAGGAGCATGGACAC-3' |
| | R (SEQ ID NO: 5) | 5'-TGAACTTGAGACACCTTTAGGAA-3' |
| | P (SEQ ID NO: 6) | 5'-CAACAGCAGTACCAATAAGCTGCAGGA-3' |
| TNF-α | F (SEQ ID NO: 7) | 5'-CCTGCAAACGGGCTATACCTT-3' |
| | R (SEQ ID NO: 8) | 5'-GTGTGGGTGAGGAGCACGTA-3' |
| | P (SEQ ID NO: 9) | 5'-CAGCCTTGGCCCTTGAAGAGGACCT-3' |
| IL-6 | F (SEQ ID NO: 10) | 5'-CCATGCAACTCATCTTGAGC-3' |
| | R (SEQ ID NO: 11) | 5'-ATGCCCATGAACCAATAAGC-3' |
| | P (SEQ ID NO: 12) | 5'-ATTTCCTGCAGTTCACCC-3' |
| CD3 | F (SEQ ID NO: 13) | 5'-CGGAGTTCGCCAGTCAAGA-3' |
| | R (SEQ ID NO: 14) | 5'-TTGGTGGTTTCCTTGAAGACG-3' |
| | P (SEQ ID NO: 15) | 5'-CTTCAGACAAGCAGACTCTGTTGCCCAA-3' |
| CD4 | F (SEQ ID NO: 16) | 5'-AGGTCTCAAAGCCCGAGAAGA-3' |
| | R (SEQ ID NO: 17) | 5'-GTAGGCACTGCCACATCCCT-3' |
| | P (SEQ ID NO: 18) | 5'-ATTCGGGTGCCAAACCCCAAGG-3' |
| CD8 | F (SEQ ID NO: 19) | 5'-TGGACTTCGCCTGTGATATCTAC-3' |
| | R (SEQ ID NO: 20) | 5'-GTTTCCGGTGGTGACAGATGA-3' |
| | P (SEQ ID NO: 21) | 5'-TGCGCGGTCCTTCTGTTGTCACTG-3' |
| CD14 | F (SEQ ID NO: 22) | 5'-AAACTCCCTGGATCTGTCATTT-3' |
| | R (SEQ ID NO: 23) | 5'-CTGACCGTGGCTTCCTATTT-3' |
| | P (SEQ ID NO: 24) | 5'-TCCCTTAGGCACTTGCTCCAACC-3' |
| CD56 | F (SEQ ID NO: 25) | 5'-AAACCATGACGGAGGGAAAC-3' |
| | R (SEQ ID NO: 26) | 5'-GACTCCGACTTTGCTTCTACAG-3' |
| | P (SEQ ID NO: 27) | 5'-ACACAGAACCCAATGAGACCACG-3' |
| CD79B | F (SEQ ID NO: 28) | 5'-ACCCTCCTCATCATCCTCTT-3' |
| | R (SEQ ID NO: 29) | 5'-CAATGTCCAGGCCCTCATAG-3' |
| | P (SEQ ID NO: 30) | 5'-ATCGTGCCCATCTTCCTGTTGCT-3' |
| 18S rRNA | F (SEQ ID NO: 31) | 5'-GTAACCCGTTGAACCCCATT-3' |
| | R (SEQ ID NO: 32) | 5'-GGGACTTAATCAACGCAAGC-3' |
| | P (SEQ ID NO: 33) | 5'-GCAATTATTCCCCATGAACG-3' |

F: forward primer; R: reverse primer; P: probe.

Liver Biopsy

Ultrasound-guided liver punch biopsies were performed on anesthetized animals using 16-gauge disposable biopsy needle kit mounted on an imaging manifold attached to an ultrasound instrument. At each sampling, 2 to 3 cores, each 16-gauge×1 to 2 cm were obtained. After the biopsy was taken, animals were prophylactically treated i.m. with long-acting benzathine penicillin

Viral Nucleic Acids

Liver biopsies were quantitatively analyzed for WHV DNA RI, WHV cccDNA, and WHV RNA (Southern and Northern blot hybridization) as described in the literature (Menne, S., et al., Antimicrob Agents Chemother, 2008. 52(10): p. 3617-32.

Disease Progression

Biopsies were assessed for liver disease progression (histology) using criteria developed for woodchuck liver sections (Peek, S. F., et al., Hepatology, 2001. 33(1): p. 254-66. Tennant, B. C., et al. Hepatology, 1998. 28(1): p. 179-91.) as well as using the METAVIR scale for scoring human liver samples. In addition, Formalin-fixed, paraffin-embedded sections stained with H&E were assessed.

Immunohistochemistry

WHcAg and WHsAg expression (immunohistochemistry) was assessed in liver biopsy samples. Formalin-fixed, paraffin-embedded sections were prepared and stained as described in the literature (Peek, S. F., et al., Hepatology, 2001. 33(1): p. 254-66. Tennant, B. C., et al. Hepatology, 1998. 28(1): p. 179-91. Cote, P. J., et al. Hepatology, 2000. 31(1): p. 190-200) and assessed.

Statistical Analysis

Intergroup statistical comparisons were performed for the following parameters: mean body weights, body temperatures, viral serological and hepatic parameters, hematology parameters, clinical chemistries, PB MC proliferative responses, and liver histology and immunohistochemistry scores.

Results from woodchucks treated with AIC649 plus ETV (Group 4) were compared to the values at T0 or pretreatment, and also to those of woodchucks treated with “Vehicle AIC” plus “Vehicle ETV” (Group 1), with AIC649 plus “Vehicle ETV” (Group 2), and with “Vehicle AIC” plus ETV (Group 3) by using unpaired Student's t-test with equal variance.

Where indicated, for geometric means, the log-transformed data for serum WHV DNA and serum WHsAg were calculated and averaged arithmetically and tested using Student's t-test. P values ≤0.05 were considered statistically significant.

Results

The bi-phasic response pattern induced by AIC649 monotherapy previously observed (Paulsen et al. (2015): PLOSOne, 10, e0144383) was confirmed. Treatment with AIC649 alone already led to a clear reduction of WHV DNA as well as WHsAg from pretreatment levels. A significant and surprisingly even stronger and sustained antiviral effect was observed in the AIC649+ETV combination group: WHV DNA and WHsAg stayed markedly suppressed or even undetectable for several months in responding animals (FIGS. 2 and 3). Cell mediated immune responses (FIG. 4), as well as anti-WHsAg antibody response (FIG. 5), were observed in the two groups receiving AIC649 but not in the ETV monotherapy group. The changes in most of the liver disease markers were comparable between the groups, but the progression of steatosis and the increases in GGT (FIG. 6) during the study appeared slower in the AIC649 alone and the AIC649+ETV combination group. Analyses of immunological parameters revealed that AIC649 treatment induces cytokines and cell markers in the liver of chronic WHV carrier woodchucks (FIG. 7). AIC649-treatment was well tolerated.

### BRIEF DESCRIPTION OF THE FIGURES

FIG. 1

Study Design

Treatment/combination (week 0-12):

AIC649 or “Vehicle AIC”: i.v. treatment, twice a week for 12 weeks ETV or “Vehicle ETV”: once daily treatment, p.o., for 12 weeks

Maintenance:

Week 13-24: AIC649 or “Vehicle AIC”: i.m. treatment, twice a week for 12 weeks

Week 25-36: AIC649 or “Vehicle AIC”: i.m. treatment, once a week for 12 weeks

ETV=Entecavir

FIG. 2

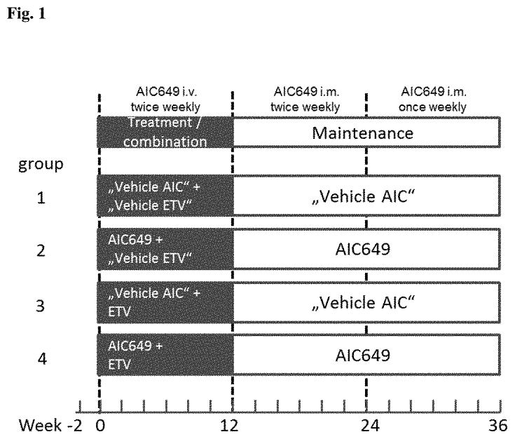

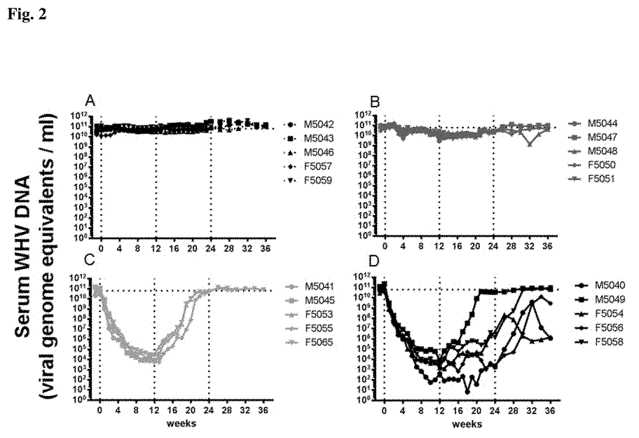

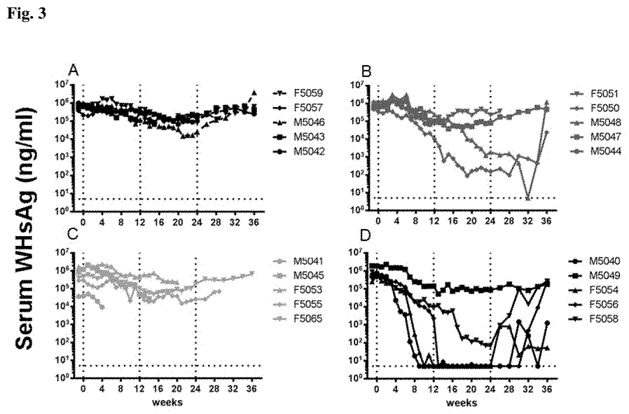

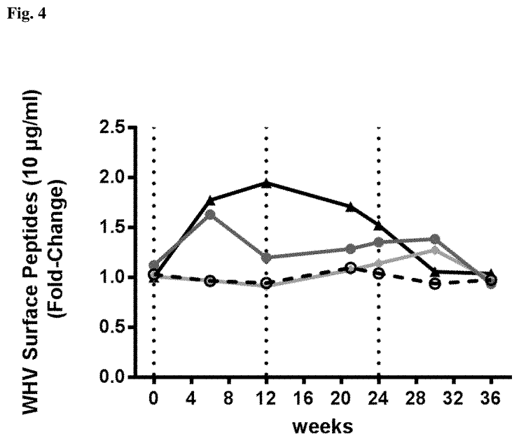

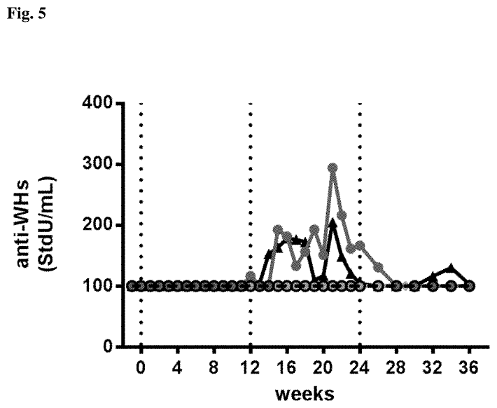

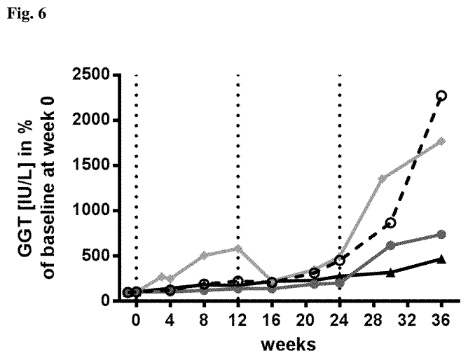

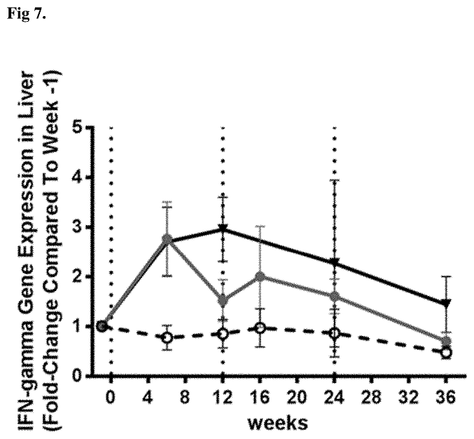

The combination of AIC649 with Entecavir leads to synergistic reduction of viremia levels in chronic WHV carrier woodchucks. AIC649, ETV or the Vehicle AIC, Vehicle ETV were administered according to the study design. At the indicated time points woodchucks were bled and serum was subjected to determination of WHV DNA load. n=5/group at start of experiment (deaths in group 1:1 animal week 21; group 2:1 animal each week 12, 26; group 3:1 animal each at week 4, 14, 21, 29; group 4: none). Horizontal dotted line representing the mean viral genome equivalents (ge/ml) of all groups at T0 (6.36×10¹⁰ viral ge/ml). Vertical dotted lines at week 0 indicated the start of treatment or changes in treatment regimen (week 12 and 24). (A) Serum WHV DNA concentrations of individual woodchucks (identified by different symbols) in group 1 (vehicle), (B) group 2 (AIC649 only); (C) group 3 (ETV only), (D) group 4 (ETV+AIC649).

FIG. 3

The combination of AIC649 with Entecavir leads to synergistic reduction of antigenemia levels in chronic WHV carrier woodchucks. AIC649, ETV or the Vehicle AIC, Vehicle ETV were administered according to the study design. At the indicated time points woodchucks were bled and serum was subjected to determination of WHsAg load. n=5/group at start of experiment (deaths in group 1:1 animal week 21; group 2:1 animal each week 12, 26; group 3:1 animal each at week 4, 14, 21, 29; group 4: none). Horizontal dotted line indicates the detection limit for WHsAg. Vertical dotted lines at week 0 indicated the start of treatment or changes in treatment regimen (week 12 and 24). (A) Serum WHsAg concentrations of individual woodchucks (identified by different symbols) in group 1 (vehicle), (B) group 2 (AIC649 only); (C) group 3 (ETV only), (D) group 4 (AIC649+ETV).

FIG. 4

Only AIC649 treatment stimulates cell mediated immunity in chronic WHV carrier woodchucks even more pronounced in combination with ETV. AIC649, ETV or the Vehicle AIC, Vehicle ETV were administered according to the study design. At the indicated time points woodchucks were bled and PBMCs were stimulated with WHsAg peptides. Values were normalized to individual baseline at T0 and are given as fold change from baseline. n=5/group at start of experiment (deaths in group 1:1 animal week 21; group 2:1 animal each week 12, 26; group 3:1 animal each at week 4, 14, 21, 29; group 4: none). Given is the geometric mean per group. Vertical dotted lines at week 0 indicated the start of treatment or changes in treatment regimen (week 12 and 24). ( [symbol] ) group 1 (vehicle), ( [symbol] ) group 2 (AIC649 only); ( [symbol] ) group 3 (ETV only), ( [symbol] ) group 4 (ETV+AIC649).

FIG. 5

Only AIC649 treatment stimulates development of anti-WHsAg antibodies in chronic WHV carrier woodchucks even more pronounced in combination with ETV. AIC649, ETV or the Vehicle AIC, Vehicle ETV were administered according to the study design. At the indicated time points woodchucks were bled and anti-WHs antibody levels in serum were quantified. Values were normalized to individual baseline at T0 and are given in %. n=5/group at start of experiment (deaths in group 1:1 animal week 21; group 2:1 animal each week 12, 26; group 3:1 animal each at week 4, 14, 21, 29; group 4: none). Given is the geometric mean per group. Vertical dotted lines at week 0 indicated the start of treatment or changes in treatment regimen (week 12 and 24). ( [symbol] ) group 1 (vehicle), ( [symbol] ) group 2 (AIC649 only); ( [symbol] ) group 3 (ETV only), ( [symbol] ) group 4 (ETV+AIC649).

FIG. 6

AIC649 treatment appears to slow down increases in GGT in chronic WHV carrier woodchucks. ETV or the Vehicle AIC, Vehicle ETV were administered according to the study design. At the indicated time points woodchucks were bled and GGT levels in serum were quantified. Values were normalized to individual baseline at T0 and are given in %. n=5/group at start of experiment (deaths in group 1:1 animal week 21; group 2:1 animal each week 12, 26; group 3:1 animal each at week 4, 14, 21, 29; group 4: none). Given is the geometric mean per group. Horizontal dotted line indicates the baseline (100%). Vertical dotted lines at week 0 indicated the start of treatment or changes in treatment regimen (week 12 and 24). ( [symbol] ) group 1 (vehicle), ( [symbol] ) group 2 (AIC649 only); ( [symbol] ) group 3 (ETV only), ( [symbol] ) group 4 (ETV+AIC649).

FIG. 7

AIC649 treatment, but not placebo treatment, induced IFN-γ in the liver of in chronic WHV carrier woodchucks. AIC649, ETV or the Vehicle AIC, Vehicle ETV were administered according to the study design. Liver biopsies were taken at the indicated time points and subjected to determination of cytokine transcript levels. Values were normalized to individual baseline at week −1. n=5/group at start of the experiment (deaths in group 1:1 animal week 21; group 2:1 animal each week 12, 26; group 3:1 animal each at week 4, 14, 21, 29; group 4: none). Results were plotted only when at least 3 values per time point were available. In group 2 (ETV only) this was only the case at week −1, therefore, no curve is available for this group. Results are given as mean fold change of the group+/−SEM. Vertical dotted lines at week 0 indicated the start of treatment or changes in treatment regimen (week 12 and 24). ( [symbol] ) group 1 (vehicle), ( [symbol] ) group 2 (AIC649 only); ( [symbol] ) group 3 (ETV only), ( [symbol] ) group 4 (ETV+AIC649).

### CONCLUSION

There were no findings of toxicological relevance associated with AIC649-treatment.
Treatment with AIC649 enhanced and extended suppression of viral nucleic acids by ETV in the liver and in blood by several months and AIC649 appeared to stimulate immune responses to WHV antigens: Cell mediated immune responses to WHV antigens were detected, as were anti-WHV antibodies and cytokine induction.
Cell mediated responses to WHsAg correlated with loss or reduction in circulating WHsAg.

AIC649 alone induced reduction of WHsAg despite apparently minor reductions in circulating WHV DNA level.

AIC649 treatment led to sustained loss of hepatic WHc or WHs antigen expression which was more frequent and more pronounced in combination with ETV, but did not occur without AIC649 treatment.

Virological parameters such as WHV DNA and WHsAg indicated a possible synergistic interaction between AIC649 and ETV.

The observed sustained loss of WHsAg and the induction of anti-WHsAg antibodies accompanied by cell mediated immune responses support the hypothesis of AIC649 inducing a physiologically “concerted”, reconstituted immune response to WHV. AIC649 as a combination partner to ETV dramatically increases the efficacy of treatment. AIC649's potential for inducing a functional cure in HBV-infected patients is supported by this preclinical study.

1. A composition comprising Parapoxvirus ovis selected from the group comprising:
optionally inactivated Parapoxvirus ovis (PPVO) virions and/or active fragments thereof, and/or
nucleic acid vectors or synthetic nucleic acid molecules expressing PPVO and/or at least one active fragment thereof, and/or
cells comprising PPVO virions or fragments thereof and/or nucleic acid vectors and/or synthetic nucleic acid molecules expressing PPVO and/or at least one active fragment thereof,
for use in combination with at least one different antiviral drug for the treatment of an individual with a Hepatitis B Virus (HBV) infection.

2. The composition comprising Parapoxvirus ovis selected from the group comprising:
optionally inactivated Parapoxvirus ovis (PPVO) virions and/or active fragments thereof, and/or
nucleic acid vectors or synthetic nucleic acid molecules expressing PPVO and/or at least one active fragment thereof, and/or
cells comprising PPVO virions or fragments thereof and/or nucleic acid vectors and/or synthetic nucleic acid molecules expressing PPVO and/or at least one active fragment thereof,
for use in combination with at least one different antiviral drug for the treatment of an individual with a Hepatitis B Virus (HBV) infection in accordance with claim 1,
wherein the different antiviral drug is an anti-HBV antiviral drug.

3. The composition comprising Parapoxvirus ovis (PPVO) for use in combination with at least one different antiviral drug for the treatment of an individual with a Hepatitis B Virus (HBV) infection in accordance with claim wherein the PPVO is a recombinant virus nucleic acid or at least one active fragment thereof, and/or wherein the PPVO is a recombinantly produced virion and/or at least one active fragment thereof.

4. The composition comprising Parapoxvirus ovis (PPVO) for use in combination with at least one different antiviral drug for the treatment of an individual with a Hepatitis B Virus (HBV) infection according to claim 1, wherein the PPVO is selected from the group of PPVO strains comprising NZ2, NZ7, NZ10, D1701, OV/20, OV/7, OV/C2, OV/mi-90, OV-Torino, SA00, Bo29, orf11, Greek orf strain 155, and/or Greek orf strain 176 or a taxonomically related Parapoxvirus ovis orf strain.

5. The composition comprising PPVO for use in combination with at least one different antiviral drug for the treatment of an individual with a HBV infection according to claim 1, wherein the antiviral drug is selected from the group of drugs comprising nucleotide/nucleoside analogues as active ingredients, Capsid assembly inhibitors or modulators, capsid/core inhibitors or modulators, encapsidation inhibitors or modulators, RNAi, Therapeutic vaccination, Toll-like-receptor (TLR) agonists and antagonists, epigenetic modifiers, entry inhibitors or modulators, cyclophilin inhibitors or modulators, Inhibitors of HBsAg secretion, HBsAg inhibitors, HBV entry inhibitors or modulators, cccDNA inhibitors, immunomodulators, particularly Interferons and other cytokines, and/or check-point inhibitors, particularly PD-1.

6. The composition comprising PPVO for use in combination with at least one different antiviral drug for the treatment of an individual with a HBV infection according to claim 1, wherein the group of drugs comprising nucleotide/nucleoside analogues as active ingredients comprises Tenofovir, Tenofovir disoproxil fumarate (TDF), Tenofovir-Alafenamid (TAF), Entecavir, Lamivudine, Telbivudine, Adefovir, Emtricitabine, and/or Clevudine.

7. The composition comprising PPVO for use in combination with at least one different antiviral drug for the treatment of an individual with a HBV infection according to claim 1, wherein PPVO and the at least one different antiviral drug are formulated for separate/subsequent administration, or wherein the PPVO and the at least one different drug as defined in any of the preceding claims are formulated for concomitant/simultaneous administration.

8. The composition comprising PPVO for use in combination with at least one different antiviral drug for the treatment of an individual with a HBV infection according to claim 1, wherein PPVO and the at least one different antiviral drug are formulated for separate/subsequent administration, or wherein the PPVO and the at least one different drug as defined in any of the preceding claims are formulated for concomitant/simultaneous administration.

9. The composition comprising PPVO for use in combination with at least one different antiviral drug for the treatment of an individual with a HBV infection according to claim 1, wherein the PPVO and the at least one different antiviral drugs are provided as single drug units or combination products selected from the group comprising: tablets, capsules, lozenges, particularly acid-resistant capsules, drops, patches, depot administration forms, solutions, solutions for injection, solution for infusion, dilutions, creams, ointments, salves, powders, powder for reconstitution, powder for reconstitution and infusion, and/or sprays.

10. The composition comprising PPVO for use in combination with at least one different antiviral drug for the treatment of an individual with a HBV infection according to claim 1, wherein said individual is selected from the group of patients with acute HBV infection, chronic HBV infection, patients with detectable HBsAg, patients with detectable HBV RNA, patients with detectable HBV DNA, patients with detectable cccDNA, patients with liver inflammation, patients with liver steatosis, patients with liver fibrosis, patients with liver cirrhosis, patients with liver cancer, patients with hepatocellular carcinoma, acutely or asymptomatically or chronically infected patients, patients subjected to antiviral treatment, patients that do not respond to antiviral treatment with antiviral drugs according to any one of claims 1 to 9, or patients that have acquired resistance to at least one antiviral drug, and/or patients that are co-infected with at least one additional pathogenic virus selected from the group comprising deltavirus, retroviridae, herpesviridae, poxviridae, parvoviridae, adenoviridae, picornaviridae, hepadnaviridae, flaviviridae, orthomyxoviridae, paramyxoviridae, papovaviridae, polyomaviridae, rhabdoviridae, coronaviridae, bunyaviridae, arenaviridae, reoviridae, and togaviridae.

11. The composition comprising PPVO for use in combination with at least one different antiviral drug for the treatment of an individual with a HBV infection according to claim 1, wherein the dose of PPVO is in the range of 1×10⁶-1×10¹⁰ viral particles and the dose of the at least one different antiviral drug is selected according to the manufacturer's instructions.

12. The composition comprising PPVO for use in combination with at least one different antiviral drug for the treatment of an individual with a HBV infection according to claim 1, wherein PPVO and the at least one different antiviral drug are administered for ≤72 weeks, preferably ≤60 weeks, more preferably ≤48 weeks, ≤36 weeks, ≤24 weeks, ≤12 weeks, ≤6 weeks, ≤4 weeks, ≤2 weeks, or ≤1 week.

13. The composition according to any of the preceding claims for use in combination with at least one different antiviral drug for the treatment of an individual with HBV infection according to claim 1, wherein the PPVO is inactivated.

14. The composition comprising PPVO for use in combination with at least one different antiviral drug for the treatment of an individual with a HBV infection according to claim 1, wherein the at least one different antiviral drug is Entecavir.

15. The composition comprising PPVO for use in combination with at least one different antiviral drug for the treatment of an individual with a HBV infection according to claim 1, wherein the patient treated with said composition and with at least one different antiviral drug is a patient that is HBsAg and/or HBeAg positive, and wherein the HBsAg and/or HBeAg load is reduced or HBsAg and/or HBeAg loss occurs over the course of the treatment as defined in any of the foregoing claims.

16. The composition comprising PPVO for use in combination with at least one different antiviral drug for the treatment of an individual with a HBV infection according to claim 1, wherein the composition is formulated for intravenous, intramuscular, oral, parenteral, topical, intradermal, and/or subcutaneous administration.

17. A method of treatment of a HBV-infected patient in need thereof with an effective amount of PPVO and an effective amount of at least one different antiviral drug, wherein the PPVO is selected from the group comprising:
optionally inactivated Parapoxvirus ovis (PPVO) virions and/or active fragments thereof, and/or
nucleic acid vectors or synthetic nucleic acid molecules expressing PPVO and/or at least one active fragment thereof, and/or
cells comprising PPVO virions or fragments thereof and/or nucleic acid vectors and/or synthetic nucleic acid molecules expressing PPVO and/or at least one active fragment thereof.

18. The method of treatment according to claim 17, wherein the PPVO is a recombinant virus nucleic acid or at least one active fragment thereof, and/or wherein the PPVO is a recombinantly produced virion and/or active fragments thereof.

19. The method according to claim 17, wherein the different antiviral drug is selected from the group of drugs comprising nucleotide/nucleoside analogues as active ingredients, Capsid assembly inhibitors or modulators, capsid/core inhibitors or modulators, encapsidation inhibitors or modulators, RNAi, Therapeutic vaccination, Toll-like-receptor (TLR) agonists and -antagonists, epigenetic modifiers, entry inhibitors or modulators, cyclophilin inhibitors or modulators, Inhibitors of HBsAg secretion, HBsAg inhibitors, HBV entry inhibitors or modulators, cccDNA inhibitors, immunomodulators, particularly Interferons and other cytokines, and/or check-point inhibitors, particularly PD-1.

20. The method according to claim 17, wherein the group of drugs comprising nucleotide/nucleoside analogues as active ingredients comprises Tenofovir, Tenofovir disoproxil fumarate (TDF), Tenofovir-Alafenamid (TAF), Entecavir, Lamivudine, Telbivudine, Adefovir, Emtricitabine, and/or Clevudine.

21. The method according to claim 17, wherein the antiviral drug is Entecavir.

22. The method according to claim 17, wherein PPVO and the at least one different antiviral drug are separately/sequentially administered.

23. The method according to claim 17, wherein PPVO and the at least one different antiviral drug are concomitantly/simultaneously administered.

24. The method according to claim 17, wherein PPVO and the at least one different antiviral drug is provided in separate single unit form or as a combination products selected from the group comprising: tablets, capsules, lozenges, particularly acid-resistant capsules, drops, patches, depot administration forms, solutions, solutions for injection, solution for infusion, dilutions, creams, ointments, salves, powders, powder for reconstitution, powder for reconstitution and infusion, and/or sprays.

25. The method according to claim 17, wherein PPVO and/or the at least one different antiviral drug are formulated for intravenous, intramuscular, oral, parenteral, topical, intradermal, and/or subcutaneous administration.

26. The method according to claim 17, wherein said individual is selected from the group of patients with acute HBV infection, chronic HBV infection, patients with detectable HBsAg, patients with detectable HBV RNA, patients with detectable HBV DNA, patients with detectable cccDNA, patients with liver inflammation, patients with liver steatosis, patients with liver fibrosis, patients with liver cirrhosis, patients with liver cancer, patients with hepatocellular carcinoma, asymptomatic or acutely or chronically infected patients, patients subjected to antiviral treatment, patients that do not respond to antiviral treatment with antiviral drugs according to any one of the preceding claims, or patients that have acquired resistance to at least one antiviral drug, patients that are co-infected with at least one additional pathogenic virus selected from the group comprising deltavirus, retroviridae, herpesviridae, poxviridae, parvoviridae, adenoviridae, picornaviridae, hepadnaviridae, flaviviridae, orthomyxoviridae, paramyxoviridae, papovaviridae, polyomaviridae, rhabdoviridae, coronaviridae, bunyaviridae, arenaviridae, reoviridae, and togaviridae.

27. The method according to claim 17, wherein the dose of PPVO is in the range of 1×10⁶-1×10¹⁰ viral particles, and/or wherein the dose of the at least one different antiviral drug is selected according to the manufacturer's instructions.

28. The method according to claim 17, wherein PPVO and the at least one different antiviral drug are administered for ≤72 weeks, preferably ≤60 weeks, more preferably ≤48 weeks, ≤36 weeks, ≤24 weeks, ≤12 weeks, ≤6 weeks, ≤4 weeks, ≤2 weeks, or ≤1 week.

29. A method for the reduction of HBV viral load in a HBV-infected patient in need thereof comprising administering an effective amount of PPVO and an effective amount of at least one different antiviral drug as defined in claim 1.

30. A method for the reduction of HBsAg load in a HBV-infected patient in need thereof comprising administering an effective amount of PPVO and an effective amount of at least one different antiviral drug as defined in claim 1.

31. A method for the reduction of liver damage, liver cirrhosis, and/or liver fibrosis, in a HBV-infected patient in need thereof comprising administering an effective amount of PPVO and an effective amount of at least one different antiviral drug as defined in claim 1.

32. A method for inducing liver tissue regeneration in a HBV-infected patient in need thereof comprising administering an effective amount of PPVO and an effective amount of at least one different antiviral drug as defined in claim 1.

33. A method for reducing side-effects associated with the treatment of a HBV-infected patient, wherein said side-effects are caused by the treatment with interferons and/or nucleotide/nucleoside analogues comprising administering an effective amount of PPVO and an effective amount of at least one different antiviral drug as defined in claim 1, in particular the reduction of side-effects selected from the group comprising fever, tissue inflammation, psychological disturbances, and/or hematological disturbances.

34. A method for the reduction of HBeAg load in a HBV-infected patient comprising administering an effective amount of PPVO and an effective amount of at least one different antiviral drug as defined in claim 1.

35. A method for the restoration and/or reactivation of the immune response in a HBV-infected patient in need thereof comprising administering an effective amount of PPVO and an effective amount of at least one different antiviral drug as defined in claim 1.

36. A method of reducing the amount of HBV DNA, eliminating HBV DNA and/or silencing HBV DNA, in particular cccDNA in a HBV-infected patient comprising administering an effective amount of PPVO and an effective amount of at least one different antiviral drug as defined in claim 1.

37. A method of preventing the de novo formation of cccDNA in a HBV-infected patient comprising administering an effective amount of PPVO and an effective amount of at least one different antiviral drug as defined in claim 1.

38. A method of inhibiting or reducing the expression of HBV proteins in a HBV-infected patient comprising administering an effective amount of PPVO and an effective amount of at least one different antiviral drug as defined in claim 1.

39. A method of suppressing replication of HBV in a HBV-infected patient comprising administering an effective amount of PPVO and an effective amount of at least one different antiviral drug as defined in claim 1.

40. A method of eradication of HBV in a HBV-infected patient comprising administering an effective amount of PPVO and an effective amount of at least one different antiviral drug as defined in claim 1.

41. A method of breaking immunological tolerance towards HBV infections in a HBV-infected patient comprising administering an effective amount of PPVO and an effective amount of at least one different antiviral drug as defined in claim 1.

42. A method of breaking tolerance towards HBsAg and/or HBeAg in a HBV-infected patient comprising administering an effective amount of PPVO and an effective amount of at least one different antiviral drug as defined in claim 1.

43. A method of inducing HBsAg-specific antibodies in a HBV-infected patient comprising administering an effective amount of PPVO and an effective amount of at least one different antiviral drug as defined in claim 1.

44. A method of inducing HBeAg-specific antibodies in a HBV-infected patient comprising administering an effective amount of PPVO and an effective amount of at least one different antiviral drug as defined in claim 1.

45. A method of slowing down or inhibiting the progression of steatosis in a HBV-infected patient comprising administering an effective amount of PPVO and an effective amount of at least one different antiviral drug as defined in claim 1.

46. A method according to claim 17, wherein PPVO and the at least one different antiviral drug are administered for ≤72 weeks, preferably ≤60 weeks, more preferably ≤48 weeks, ≤24 weeks, ≤12 weeks, ≤6 weeks, ≤4 weeks, ≤2 weeks, or ≤1 week.

47. A medicinal kit product comprising a first container comprising a pharmaceutical compositions comprising PPVO, preferably inactivated PPVO, and a second container comprising a pharmaceutical compositions comprising at least one different antiviral drug as defined in any one of the preceding claims or a pharmaceutical composition comprising PPVO, preferably inactivated PPVO, and at least one different antiviral drug as defined in claim 1 in form of a combined formulation, and optionally instructions for use, pharmaceutically acceptable media for reconstitution, syringes, and/or microneedles.

48. The medicinal kit product according to claim 47, wherein the compositions comprising PPVO, preferably inactivated PPVO, and the pharmaceutical composition comprising at least one different antiviral drug are formulated as tablets, capsules, lozenges, particularly acid-resistant capsules, drops, patches, depot administration forms, solutions, solutions for injection, solution for infusion, dilutions, creams, ointments, salves, powders, powder for reconstitution, powder for reconstitution and infusion, and/or sprays.


SEQUENCE LISTING (33 sequences)

SEQ ID NO: 1 - DNA, 20 bp, Marmota marmota
ctcaagctgttgctgtcctc

SEQ ID NO: 2 - DNA, 20 bp, Marmota marmota
cttctgggtgctgaagaggt

SEQ ID NO: 3 - DNA, 23 bp, Marmota marmota
ccagatgacccagcagatcctca

SEQ ID NO: 4 - DNA, 20 bp, Marmota marmota
atccaaaggagcatggacac

SEQ ID NO: 5 - DNA, 23 bp, Marmota marmota
tgaacttgagacacctttaggaa

SEQ ID NO: 6 - DNA, 27 bp, Marmota marmota
caacagcagtaccaataagctgcagga

SEQ ID NO: 7 - DNA, 21 bp, Marmota marmota
cctgcaaacgggctatacctt

SEQ ID NO: 8 - DNA, 20 bp, Marmota marmota
gtgtgggtgaggagcacgta

SEQ ID NO: 9 - DNA, 25 bp, Marmota marmota
cagccttggcccttgaagaggacct

SEQ ID NO: 10 - DNA, 20 bp, Marmota marmota
ccatgcaactcatcttgagc

SEQ ID NO: 11 - DNA, 20 bp, Marmota marmota
atgcccatgaaccaataagc

SEQ ID NO: 12 - DNA, 18 bp, Marmota marmota
atttcctgcagttcaccc

SEQ ID NO: 13 - DNA, 19 bp, Marmota marmota
cggagttcgccagtcaaga

SEQ ID NO: 14 - DNA, 21 bp, Marmota marmota
ttggtggtttccttgaagacg

SEQ ID NO: 15 - DNA, 28 bp, Marmota marmota
cttcagacaagcagactctgttgcccaa

SEQ ID NO: 16 - DNA, 21 bp, Marmota marmota
aggtctcaaagcccgagaaga

SEQ ID NO: 17 - DNA, 20 bp, Marmota marmota
gtaggcactgccacatccct

SEQ ID NO: 18 - DNA, 22 bp, Marmota marmota
attcgggtgccaaaccccaagg

SEQ ID NO: 19 - DNA, 23 bp, Marmota marmota
tggacttcgcctgtgatatctac

SEQ ID NO: 20 - DNA, 21 bp, Marmota marmota
gtttccggtggtgacagatga

SEQ ID NO: 21 - DNA, 24 bp, Marmota marmota
tgcgcggtccttctgttgtcactg

SEQ ID NO: 22 - DNA, 22 bp, Marmota marmota
aaactccctggatctgtcattt

SEQ ID NO: 23 - DNA, 20 bp, Marmota marmota
ctgaccgtggcttcctattt

SEQ ID NO: 24 - DNA, 23 bp, Marmota marmota
tcccttaggcacttgctccaacc

SEQ ID NO: 25 - DNA, 20 bp, Marmota marmota
aaaccatgacggagggaaac

SEQ ID NO: 26 - DNA, 22 bp, Marmota marmota
gactccgactttgcttctacag

SEQ ID NO: 27 - DNA, 23 bp, Marmota marmota
acacagaacccaatgagaccacg

SEQ ID NO: 28 - DNA, 20 bp, Marmota marmota
accctcctcatcatcctctt

SEQ ID NO: 29 - DNA, 20 bp, Marmota marmota
caatgtccaggccctcatag

SEQ ID NO: 30 - DNA, 23 bp, Marmota marmota
atcgtgcccatcttcctgttgct

SEQ ID NO: 31 - DNA, 20 bp, Marmota marmota
gtaacccgttgaaccccatt

SEQ ID NO: 32 - DNA, 20 bp, Marmota marmota
gggacttaatcaacgcaagc

SEQ ID NO: 33 - DNA, 20 bp, Marmota marmota
gcaattattccccatgaacg